(19) United States
(12) Patent Application Publication
(10) Pub. No.: US 2024/0335293 A1
(43) Pub. Date: Oct. 10, 2024

(54) IMPLANTATION SYSTEM

(71) Applicant: Aesculap AG, Tuttlingen (DE)

(72) Inventors: Thomas Saueressig, Tuttlingen (DE); Philipp Hauff, Tuttlingen (DE); Sebastian Dziuba, Hemmingen (DE)

(21) Appl. No.: 18/747,530

(22) Filed: Jun. 19, 2024

Related U.S. Application Data
(63) Continuation of application No. PCT/EP2022/086996, filed on Dec. 20, 2022.

(30) Foreign Application Priority Data
Dec. 23, 2021 (DE) 10 2021 134 527.8

(51) Int. Cl. A61F 2/36

(52) U.S. Cl. CPC A61F 2/367; A61F 2/3676; A61F 2220/0025

(57) ABSTRACT

An implantation system includes a prosthesis shaft, such as a femoral shaft of a hip joint endoprosthesis, for insertion into a cavity of a bone. The prosthesis shaft has a proximal end and a distal end and defines a prosthesis shaft longitudinal axis. A shaft coupling element is formed at the proximal end for coupling to a first handling instrument for inserting the prosthesis shaft into the cavity and/or for extracting it therefrom. The implantation system includes an adapter apparatus with a first adapter device and a second adapter device. The first adapter device is configured to releasably couple to the shaft coupling element in force-locking and/or positive-locking manner in a coupling position. The second adapter device is configured to releasably couple to a second handling instrument. The second adapter device is also arranged or formed on the adapter apparatus laterally offset relative to the first adapter device.

### CROSS-REFERENCE TO RELATED APPLICATIONS

This application is a continuation of International Application No. PCT/EP2022/086996, filed on Dec. 20, 2022, and claims priority to German Application No. 10 2021 134 527.8, filed on Dec. 23, 2021. The contents of International Application No. PCT/EP2022/086996 and German Application No. 10 2021 134 527.8 are incorporated by reference herein in their entireties.

### FIELD

The present disclosure relates to an implantation system comprising a prosthesis shaft, in particular a femoral shaft of a hip joint endoprosthesis, for insertion into a cavity of a bone, wherein the prosthesis shaft has a proximal end and a distal end and defines a prosthesis shaft longitudinal axis, wherein a shaft coupling element is formed at the proximal end for coupling to a first handling instrument for inserting the prosthesis shaft into the cavity and/or for extracting it therefrom.

### BACKGROUND

Prosthesis shafts of the kind described at the outset are implanted, for example, as revision shafts of hip joint endoprostheses. This means, in particular, that a patient who is implanted with such a hip joint endoprosthesis has already had an artificial hip joint. After removing the previously implanted prosthesis shaft, the cavity in the bone, in this case in a femoral bone, must be adapted for the prosthesis shaft of the revision prosthesis. The prosthesis shaft itself is then coupled to a first handling instrument in the form of a proximal driving-in/driving-out instrument. It is known here, in particular, to screw the driving instrument, which has a short threaded portion at its distal end, to the shaft coupling element formed at the proximal end of the prosthesis shaft, which shaft coupling element may be configured in the form of an internally threaded portion.

Known driving-in instruments for this purpose are large, heavy, and have a large shaft diameter, in particular a shaft diameter that is larger than a diameter defined by the shaft coupling element. Therefore, it is not simple to use the driving-in instrument to extract the prosthesis shaft. The placement of the threaded portion at the distal end of the driving-in instrument into the internal thread on the prosthesis shaft is usually difficult for a surgeon. Soft tissue presses laterally on the driving-in instrument, such that the threaded portions often get tilted. In the worst case, it can happen that the internally threaded portion on the prosthesis shaft is irreversibly damaged, so that the extraction of the prosthesis shaft from the cavity of the bone of the patient is practically no longer possible in an intended manner with the driving-in instrument, which can also be used for extracting.

### SUMMARY

Therefore, it is an object of the present disclosure to improve a handling of the implantation system.

The object is achieved, in accordance with the present disclosure, in an implantation system of the kind described at the outset in that it comprises an adapter apparatus with a first adapter device and a second adapter device, in that the first adapter device is configured to releasably couple to the shaft coupling element in force-locking and/or positive-locking manner in a coupling position, in that the second adapter device is configured to releasably couple to a second handling instrument, and in that the second adapter device is arranged or formed on the adapter apparatus laterally offset relative to the first adapter device.

In particular, the proposed adapter apparatus makes it possible to couple its first adapter device to the shaft coupling element in a simple manner. Thus, in particular, the adapter apparatus can be formed small and compact, so that it can be easily guided by a surgeon through the tissue of the patient to the proximal end of the prosthesis shaft and coupled to the shaft coupling element thereof. The laterally offset arrangement of the second adapter device further has the advantage, in particular, that a different handling instrument than previously can be used to drive in the prosthesis shaft or to extract it from the cavity of the bone. In particular, a different coupling mechanism than the coupling mechanism between the described first handling instrument and the shaft coupling instrument can thus be used. For example, a screw connection can be dispensed with here, such that it is significantly simpler to bring the second handling instrument into engagement with the second adapter device of the adapter apparatus. For example, a latching and/or snapping connection device or a bayonet connection device may be used here. In addition, it is not necessary to screw the second handling instrument as a whole to the prosthesis shaft. For example, a screw or bayonet connection device may be provided for coupling the first adapter device to the shaft coupling instrument. For this purpose, either the surgeon can engage directly on the adapter device or can couple the adapter apparatus and the prosthesis shaft to one another with a suitable instrument that is easier to be guided through the tissue of the patient.

It is favorable if the first adapter device defines a first adapter longitudinal axis, if the second adapter device defines a second adapter longitudinal axis, and if the first adapter longitudinal axis and the second adapter longitudinal axis enclose an adapter angle. This configuration makes it possible, in particular, to arrange or form the second adapter device on the adapter apparatus such that the first and the second adapter longitudinal axis do not extend in parallel offset from one another, but instead are inclined relative to one another and enclose the adapter angle. Thus, for example, the second handling instrument can be coupled obliquely to the second adapter device with respect to the prosthesis shaft longitudinal axis in order to drive in the prosthesis shaft or extract it from the cavity of the bone.

The adapter angle preferably has a value in a range of about 15° to about 60°. In particular, the value may be in a range of about 30° to about 45°. Adapter angles in the specified ranges allow for an optimized access of the second handling instrument to the adapter apparatus coupled to the prosthesis shaft.

It is advantageous if the shaft coupling element comprises an internally threaded portion and if the first adapter device comprises an externally threaded portion corresponding to the internally threaded portion. This configuration makes it possible, in particular, to screw the adapter apparatus to the shaft coupling element by means of the first adapter device. A screw connection is thus also established here, as in the known first handling instrument described at the outset. However, as explained, this is simpler because the adapter apparatus can be formed significantly shorter overall than the first handling instrument, so that it is simpler to bring up against and screw to the prosthesis shaft and the risk of damage to the internally threaded portion is reduced.

According to a further preferred embodiment of the present disclosure, provision may be made that the first adapter device comprises an adapter element receptacle and an adapter element, and that in a separating position, in which the adapter apparatus and the prosthesis shaft are out of engagement, the adapter element is movable relative to the adapter element receptacle. In particular, it may be displaceable and/or rotatable. The proposed further development makes it possible, in particular, to bring the adapter apparatus against the prosthesis shaft and then to establish a connection by displacing and/or rotating the adapter element relative to the adapter element receptacle. For example, the adapter element can be screwed to the internally threaded portion of the prosthesis shaft. The first adapter device may be configured, in particular, in such a way that the adapter element is completely releasable from the adapter element receptacle. This makes it possible, in particular, to use a component available in an operating room as an adapter element of the first adapter device, so that the number of required parts of the implantation system can be minimized. For example, as an adapter element, a suitable sleeve-shaped separating element, which is provided for disassembling a modular prosthesis shaft into its constituent parts, can be introduced into the adapter element receptacle and brought into engagement with the shaft coupling element, in particular by screwing, for coupling the adapter apparatus to the prosthesis shaft.

It is favorable if the adapter element has a distal and a proximal end and defines an adapter element longitudinal axis and if in the coupling position the first adapter longitudinal axis and the adapter element longitudinal axis are oriented coaxially with one another. This configuration makes it possible, in particular, to couple the adapter apparatus to the prosthesis shaft, for example to screw it thereto, in a simple and defined manner. In particular, the adapter apparatus may be configured in such a way that it is able to be placed against the proximal end of the prosthesis shaft, the adapter element thereby then also being able to be guided in the adapter element receptacle in the direction of the first adapter longitudinal axis for connecting to the shaft coupling element.

The implantation system can be formed in a simple manner if the adapter element is configured in the form of a screw element and if the externally threaded portion corresponding to the internally threaded portion is formed on the adapter element commencing from its distal end or in the vicinity thereof. Such an adapter element can be screwed to an internally threaded portion of the shaft coupling element in a simple manner. Thus, in the case of such a configuration, only the adapter element is screwed to the prosthesis shaft. The adapter apparatus itself is not rotated or twisted relative to the prosthesis shaft. As described, tilting of the threaded portions to be screwed to one another can thus be prevented in a simple manner.

Preferably, the distal end of the adapter element in the coupling position projects distally out of the adapter element receptacle. This configuration makes it possible, in particular, to screw the adapter element to the prosthesis shaft in such a way that the adapter apparatus is held on the prosthesis shaft in the coupling position.

It is advantageous if arranged or formed at the proximal end of the adapter element is a tool element pointing in the proximal direction for being brought into rotationally-fixed engagement with a distal end of a screw-in tool. Such an adapter element can be manipulated in a simple and desired manner by a surgeon for coupling to the prosthesis shaft. In particular, a screw-in tool may be used here that only needs to displace a small amount of body tissue. For example, it may have a significantly thinner shaft compared to the driving-in tool described above, since it does not have to transmit driving-in or extraction forces to the prosthesis shaft.

The handling of the implantation system can be simplified, in particular, by the tool element being configured in the form of a tool element receptacle open pointing in the proximal direction. In particular, the tool element receptacle may be configured in the form of an internal polygon or an internal multilobular profile. The tool element receptacle can thus be brought into engagement with a correspondingly formed distal end of the screw-in tool in a simple and secure manner.

Further, it may be favorable if the proximal end of the adapter element projects proximally out of the adapter element receptacle. In particular, it may project beyond the proximal end of the adapter apparatus. Such an adapter element can also be manipulated manually by a surgeon, for example. In other words, they can screw the adapter element, for example, to the prosthesis shaft, namely without a screw-in tool, but instead only by grasping the proximal end of the adapter element with their hand.

For coupling the adapter apparatus to the prosthesis shaft, in particular in a clamping manner, it is favorable if the adapter element receptacle comprises a perforation formed on the adapter apparatus. The adapter element can thus pass through the adapter element receptacle and engage on a stop on the adapter apparatus, said stop comprising a stop face pointing in the distal direction. Thus, the adapter element can prevent a movement of the adapter apparatus in the proximal direction when the adapter element is connected to the prosthesis shaft.

In principle, it is possible, as already described above, to configure the adapter apparatus in such a way that the adapter element can be completely separated from the adapter apparatus. However, in order to further improve the handling of the implantation system, it is advantageous if the adapter element is held captive on the adapter apparatus. The adapter element in this case is still arranged or formed so as to be movable relative to the adapter apparatus, but cannot be separated from the adapter apparatus without damaging or destroying the adapter element and/or the adapter apparatus. In particular, it can thus be prevented that the adapter element can detach from the adapter apparatus in an undesirable manner and then, in particular, get lost in the surgical site in the patient's body.

The adapter element can be held captive on the adapter apparatus in a simple manner if it has an undercut and if a retaining projection of the adapter apparatus engages into the undercut.

It is favorable if the undercut is configured in the form of an annular groove open in the radial direction relative to the adapter element longitudinal axis and if the retaining projection is configured in the form of an annular flange pointing in the radial direction toward the adapter element longitudinal axis. The annular groove and the annular flange may be dimensioned, in particular, such that both a rotation of the adapter element relative to the adapter device and a movement of the adapter element relative to the adapter apparatus in parallel to the adapter element longitudinal axis is possible. In particular, this allows the adapter apparatus to be screwed to the prosthesis shaft.

Holding the adapter element captive on the adapter apparatus can be achieved in a simple manner, in particular, in that the adapter element is of multi-part, in particular two-part configuration, and comprises a first adapter element part and a second adapter element part, in that the undercut is delimited both by the first adapter element and by the second adapter element, and in that the adapter element parts are non-releasably connected to one another. In particular, they may be adhesively bonded or welded. The described configuration makes it possible, in particular, to couple the adapter element to the adapter apparatus without the adapter element or the adapter apparatus having to be deformed in any way to insert the retaining projection into the undercut.

For the handling of the implantation system, it is favorable if the adapter element has a head and if the head forms the proximal end of the adapter element. In particular, the head can form a stop on which the adapter apparatus is supported in the coupling position.

Preferably, an outer diameter of the head is larger than an outer diameter of the externally threaded portion. Thus, the adapter element can be supported with the head on the adapter apparatus in a simple and secure manner in order to hold said adapter apparatus in the coupling position on the prosthesis shaft in a clamping manner.

The adapter apparatus can be configured particularly compactly if the undercut is arranged or formed on the head.

Furthermore, it is favorable if the adapter element receptacle has a head receptacle portion with an engagement opening and if the engagement opening is dimensioned such that the head is movably held in the head receptacle portion. Such an adapter element receptacle makes it possible, in particular, to access the adapter element with a screw-in instrument, for example. However, if the engagement opening is dimensioned such that the head is held in the head receptacle portion, the head cannot exit the adapter element receptacle through the engagement opening. Thus, the adapter element can be secured captively to the adapter apparatus in the manner described. In other words, the engagement opening is dimensioned such that the head of the adapter element cannot pass through the engagement opening without deforming the head or the adapter apparatus.

It is advantageous if the head receptacle portion comprises an outer delimiting wall, which extends over a circumferential angle of more than 180° relative to the first adapter longitudinal axis. In particular, the circumferential angle may be more than 200°. In particular, an engagement opening can thus be formed through which the head of the adapter element can be accessed. However, the proposed delimiting wall does not allow the head to pass through the engagement opening if the outer delimiting wall extends over a circumferential angle of more than 180º. The insertion of the head into the head receptacle portion can take place, for example, by first deforming the outer delimiting wall such that the head fits through the engagement opening, and then by deforming it again such that the head is no longer able to pass through the engagement opening.

It is favorable if the head and the head receptacle portion are configured in such a way that the head is insertable in a direction parallel to the adapter longitudinal axis through the engagement opening into the head receptacle portion only in a defined rotational position relative to the first adapter longitudinal axis. In such a configuration, the head and the engagement opening are configured in the manner of a key-keyhole principle. The head must be brought into a certain rotational position in order to be inserted through the engagement opening into the head receptacle portion in parallel to the first adapter longitudinal axis. When it is then accommodated in the head receptacle portion, the head can be secured therein by rotating about the adapter longitudinal axis. Thus, the adapter element can also be temporarily secured in the adapter element receptacle, similarly to a key in a keyhole.

The implantation system can be formed in a simple manner if the engagement opening defines a non-round inner cross section and if an outer contour of the head is configured corresponding to the non-round inner cross section. This makes it possible, in particular, to insert the head in the manner of a key into a keyhole and to secure it in the head receptacle portion by rotating about the first adapter longitudinal axis.

It is favorable if the implantation system comprises an anti-rotation device for holding the adapter apparatus on the prosthesis shaft in a rotationally-fixed manner in the coupling position. The provision of such an anti-rotation device makes it possible, in particular, to manipulate the prosthesis shaft in a defined manner, either directly with the adapter apparatus or indirectly by means of a second handling instrument that is coupled to the second adapter device of the adapter apparatus.

The implantation system can be formed in a simple manner if the anti-rotation device comprises first and second anti-rotation elements, which are arranged or formed on the prosthesis shaft on the one hand and on the adapter apparatus on the other hand, and if the first and second anti-rotation elements are in engagement in the coupling position for preventing a rotation of the adapter apparatus and the prosthesis shaft relative to one another about the prosthesis shaft longitudinal axis. Thus, in particular, by means of a positive-locking connection of the first and second anti-rotation elements in the coupling position, a rotationally secured handling of the prosthesis shaft with the adapter apparatus or a second handling instrument coupled to the adapter apparatus can be achieved.

It is advantageous if the second adapter device comprises an adapter end face pointing away from the adapter apparatus in the direction of the second adapter longitudinal axis and a coupling projection projecting from the adapter end face. A second adapter device configured in that way makes it possible, in particular, to create a coupling with a second handling instrument, which is used, e.g., as a rasp handle in order to guide a rasp shaft for preparing the cavity. Such a suitable second handling instrument is known, for example, from DE 10 2008 064 518 Al and in this publication is referred to as a surgical rasp handle. The coupling projection may be configured identically to a coupling projection on the rasp shaft, also referred to as a rasp body in DE 10 2008 064 518 A1. Thus, the second handling instrument can be selectively used to couple to the adapter apparatus or to a rasp shaft. In particular, a number of instruments required for a surgical procedure can be minimized in this way.

Preferably, the coupling projection in a section plane perpendicular to the second adapter longitudinal axis has a cross sectional shape deviating from a circular shape and has a coupling recess open pointing in a direction transverse relative to the second adapter longitudinal axis. Such a coupling projection can, in particular, be coupled to a movable coupling member on the second handling instrument in a defined manner and secured against rotation. The anti-rotation function is achieved here by the outer contour of the coupling projection with its cross sectional shape deviating from a circular shape. The movable coupling member on the second handling instrument, in particular, for coupling same to the coupling projection can engage into the coupling recess and for releasing the second handling instrument from the adapter apparatus can be moved back out of the coupling recess. For this purpose, a corresponding actuation mechanism may be provided on the second handling instrument.

In accordance with a further preferred embodiment of the present disclosure, provision may be made that a laterally projecting prosthesis neck is formed at the proximal end of the prosthesis shaft, said prosthesis neck defining a prosthesis neck longitudinal axis inclined relative to the prosthesis shaft longitudinal axis by a neck angle. Such a prosthesis neck makes it possible, in particular, to connect the prosthesis shaft to a plurality of further components, for example a joint head if the prosthesis shaft is configured in the form of a femoral shaft of a hip joint endoprosthesis. The neck angle allows a form of the natural femur to be reproduced.

To be able to optimally adapt to the physiology of different patients, it is advantageous if the neck angle has a value in a range of about 20° to about 60°.

A free end of the prosthesis neck is favorably configured in the form of a coupling cone for coupling to a joint head or a neck extension. This makes it possible, in particular, to optimally adapt the hip joint endoprosthesis to the physiology of a patient by the prosthesis neck optionally being extended or being coupled to an optimally suited joint head.

Furthermore, in a further preferred form of the present disclosure, it may be advantageous if the prosthesis shaft has a longitudinal perforation extending from its distal end up to its proximal end. Such a sleeve-shaped prosthesis shaft makes it possible, in particular, to couple said prosthesis shaft to a shaft extension in a simple manner.

Preferably, the first adapter longitudinal axis extends in parallel to the prosthesis shaft longitudinal axis or is defined thereby. Such a configuration assists a surgeon in the handling of the prosthesis shaft. In particular, an orientation for coupling the prosthesis shaft to the adapter apparatus is thus predetermined.

So that the implantation system is optimally usable, in particular for revision procedures, it is favorable if it comprises at least one shaft extension that is coupleable to the distal end of the prosthesis shaft. Thus, the prosthesis shaft can be extended in the desired manner, in particular in order to secure the prosthesis shaft to the bone additionally with locking elements, such as, for example, fastening elements engaging transversely into the bone through the shaft extension or the prosthesis shaft. This is favorable, in particular, if the amount of bone substance present after explantation of a previously implanted prosthesis is no longer sufficient to insert a short prosthesis shaft, in particular without bone cement.

It is advantageous if the implantation system comprises a first handling instrument in the form of a driving-in and driving-out instrument with an instrument shaft. Such a driving-in and driving-out instrument can be used, in particular, to handle the prosthesis shaft upon insertion into the cavity. Here, the prosthesis shaft can be coupled to the first handling instrument, in particular outside the patient's body, such that the size and weight of the patient do not particularly matter here initially.

The first handling instrument can be coupled to the prosthesis shaft in a simple manner if the instrument shaft comprises a distal end with an externally threaded portion corresponding to the internally threaded portion.

The outer diameter of the instrument shaft is preferably larger than an outer diameter of the externally threaded portion. A stable connection between the first handling instrument and the prosthesis shaft can thus be established. In particular, driving-in and driving-out forces can thus be transmitted with the first handling instrument to the prosthesis shaft in an optimal manner.

In accordance with a further preferred embodiment of the present disclosure, provision may be made that the implantation system comprises a rasp for preparing the cavity. With such a rasp, which, in particular, can be adapted in its shape to a contour of the prosthesis shaft, the cavity can be prepared in a suitable manner for placing the prosthesis shaft in the bone in a defined and secure manner.

It is favorable if the rasp comprises a rasp shaft and a second handling instrument, and if in a rasp position the rasp shaft and the second handling instrument are in engagement in a force-locking and/or positive-locking manner. Such a rasp is thus of at least two-part configuration. A surgeon can use the second handling instrument, in particular, to connect it to the rasp shaft corresponding to the prosthesis shaft to be implanted in order to be able to prepare the cavity in the bone in the optimal manner. The second handling instrument can also optionally be used to couple it to the second adapter device of the adapter apparatus. Thus, the second handling instrument can selectively be used as a so-called rasp handle, as described in DE 10 2008 064 518 A1, for handling the rasp shaft or the rasp body as well as for handling the prosthesis shaft. In particular when such a second handling instrument is comprised by the implantation system, the first handling instrument described can be completely dispensed with.

It is favorable if a rasp adapter is arranged or formed on the rasp shaft and if the second handling instrument comprises a rasp adapter receptacle configured corresponding to the rasp adapter. This configuration makes it possible, in particular in a simple manner, to bring the second handling instrument and the rasp shaft into engagement with one another in a force-locking and or positive-locking manner in the rasp position, namely by the rasp adapter being brought into engagement with the rasp adapter receptacle in a force-locking and/or positive-locking manner.

Preferably, the rasp adapter is configured in such a way that is corresponds or substantially corresponds in its shape and size to the second adapter device. In other words, an identical second adapter device can be arranged or formed both on the rasp shaft and on the adapter apparatus, such that the adapter apparatus or a rasp shaft can be selectively coupled with one single second handling instrument.

In accordance with a further preferred embodiment of the present disclosure, provision may be made that the implantation system comprises a screw-in instrument that cooperates with the first adapter device and has a screw-in instrument shaft defining a screw-in tool longitudinal axis. Such a screw-in instrument makes it possible, in particular, to couple the adapter apparatus with the first adapter device, as already described in detail above, to the shaft coupling element of the prosthesis shaft. In particular, the screw-in instrument may be configured to be brought into force-locking and/or positive-locking engagement with the adapter element of the first adapter device in order to screw the adapter element, for example, to the internally threaded portion, which is comprised by the shaft coupling element.

In order to further improve the handling of the implantation system, it is favorable if an outer diameter of the screw-in instrument shaft is smaller than an outer diameter of the externally threaded portion. In particular, the outer diameter of the outer instrument shaft is at most about half as large as the outer diameter of the externally threaded portion. Thus, in particular, a particularly slim and light screw-in instrument can be provided, which can be handled sensitively by a surgeon in order to, for example, screw the adapter element to the shaft coupling element. Such a slim screw-in instrument shaft can be introduced through tissue of the patient and brought up to the prosthesis shaft without significant impairments.

Preferably, a distal end of the screw-in instrument is configured to be brought into force-locking and/or positive-locking engagement with the tool element of the adapter element. In this way, the adapter element can be handled with the screw-in instrument in a simple and secure manner.

### BRIEF DESCRIPTION OF THE DRAWING FIGURES

The subsequent description of a preferred embodiment of the present disclosure serves in conjunction with the drawing figures for further explanation.

FIG. 1 shows a schematic depiction of a first embodiment of an implantation system;

FIG. 2 shows an enlarged, partially broken view of a prosthesis shaft with an adapter apparatus coupled thereto and with a handling apparatus coupled to said adapter apparatus;

FIG. 3 shows a view similar to FIG. 2 with a screw-in instrument coupled to an adapter element of the adapter apparatus;

FIG. 4 shows an exploded depiction of the arrangement from FIG. 3 from a different perspective;

FIG. 5 shows a section view along line 5-5 in FIG. 2;

FIG. 6 shows a section view of the adapter apparatus from FIG. 7 along line 6-6;

FIG. 7 shows a view of the arrangement from FIG. 6 in the direction of the arrow A;

FIG. 8 shows a view of the arrangement from FIG. 6 in the direction of the arrow B;

FIG. 9 shows a section view along line 9-9 in FIG. 7;

FIG. 10 shows a view similar to FIG. 2 with a further embodiment of an adapter apparatus;

FIG. 11 shows a section view along line 11-11 in FIG. 10;

FIG. 12 shows a section view of the adapter apparatus from FIG. 13 along line 12-12;

FIG. 13 shows a view of the adapter apparatus from FIG. 12 in the direction of the arrow C;

FIG. 14 shows a view of the adapter apparatus from FIG. 13 in the direction of the arrow D;

FIG. 15 shows a view similar to FIG. 2 with a further embodiment of an adapter apparatus;

FIG. 16 shows a section view along line 16-16 in FIG. 15;

FIG. 17 shows a perspective view of an adapter apparatus before the insertion of an adapter element into an adapter element receptacle of the adapter apparatus;

FIG. 18 shows a view similar to FIG. 17 after the insertion of the adapter element into the adapter element receptacle;

FIG. 19 shows a view of the adapter apparatus from FIG. 19 in the direction of the arrow H in FIG. 21;

FIG. 20 shows a view of the adapter apparatus from FIG. 18 in the direction of the arrow G in FIG. 21;

FIG. 21 shows a view of the adapter apparatus from FIG. 18 in the direction of the arrow F in FIG. 19;

FIG. 22 shows a view similar to FIG. 21, but with an adapter element rotated about its longitudinal axis compared to FIG. 21;

FIG. 23 shows a view similar to FIG. 2 with a further embodiment of an adapter apparatus;

FIG. 24 shows a section view along line 24-24;

FIG. 25 shows a view of an exploded depiction of the adapter apparatus from FIG. 24 in the direction of the arrow I in FIG. 26; and

FIG. 26 shows a section view of the adapter apparatus from FIG. 25 along line 26-26 and an adapter element separated from said adapter apparatus.

### DETAILED DESCRIPTION

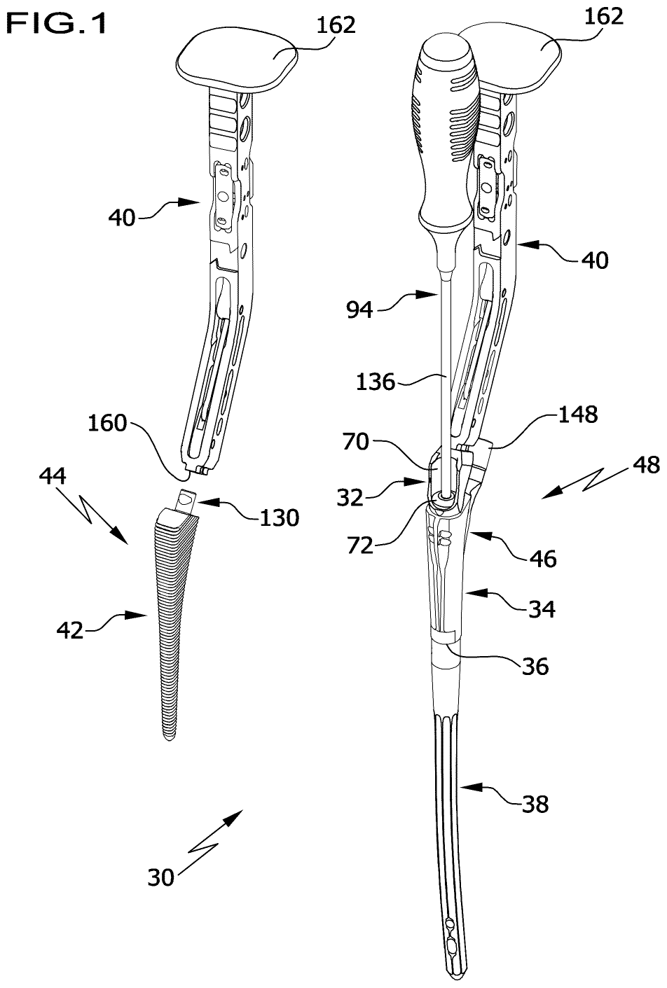

An embodiment of an implantation system is schematically depicted in FIG. 1 and is denoted as a whole with the reference numeral 30. The implantation system 30 comprises as an essential component an adapter apparatus 32, which is configured to releasably couple to a prosthesis shaft 34 in a force-locking and/or positive-locking manner.

The embodiment of the prosthesis shaft 34 depicted schematically in FIG. 1 is shown with a shaft extension 38 coupled to a distal end 36 of said prosthesis shaft.

A further optional component of the implantation system 30 is a handling instrument 40, which is configured to be releasably coupleable to the adapter apparatus 32.

For explanatory illustration, a further handling instrument 40 is depicted in FIG. 1, which is coupleable to a rasp shaft in a force-locking and/or positive-locking manner for forming a rasp 44 with which a cavity in the bone into which the prosthesis shaft 34 is to be inserted can be prepared. In a rasp position, the rasp shaft 42 and the handling instrument 40 are in force-locking and/or positive-locking engagement. In FIG. 1, a position is shown in which the handling instrument 40 and the rasp shaft 42 are separated from one another.

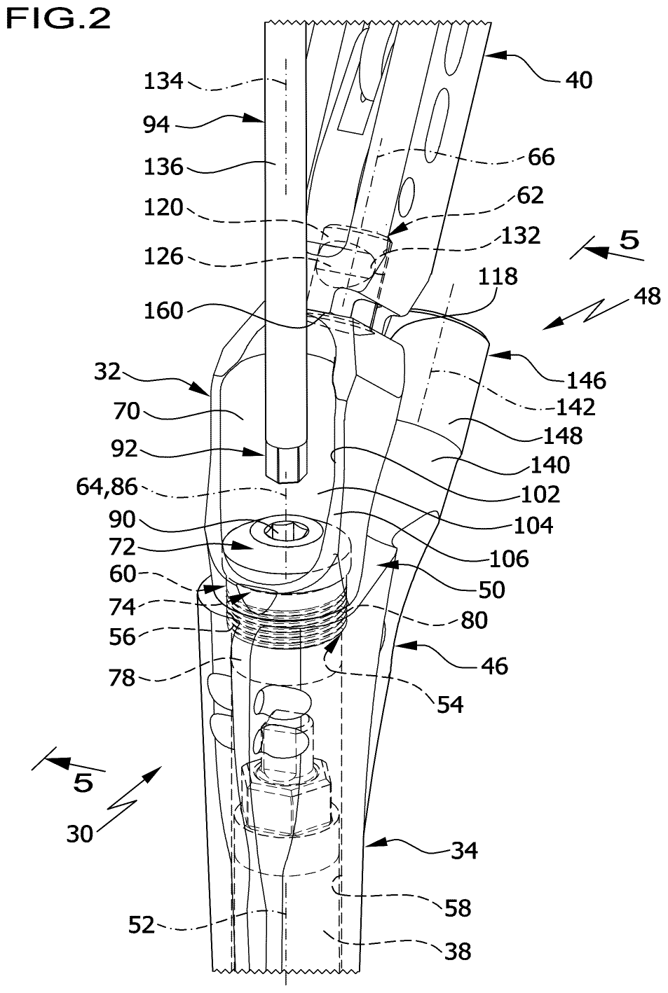

The prosthesis shaft 34 depicted schematically in FIGS. 1 to 5 is shown in the form of a femoral shaft 46 of a hip joint endoprosthesis 48. The prosthesis shaft 34 further comprises a proximal end 50. It further defines a prosthesis shaft longitudinal axis 52, which extends in a direction connecting the proximal end 50 and the distal end 36.

A shaft coupling element 54 is formed on the prosthesis shaft 34 at the proximal end 50. It comprises a short internally threaded portion 56, which extends commencing from the proximal end 50 into a longitudinal perforation 56 of the prosthesis shaft 34. In the embodiment of the prosthesis shaft 34 depicted in the Figures, the longitudinal perforation 58 extends from the distal end 36 of said prosthesis shaft up to its proximal end 50.

The internally threaded portion 56 is configured corresponding to an externally threaded portion of a handling instrument not shown in the Figures for inserting the prosthesis shaft 34 into the cavity and/or for extracting it therefrom. Such a handling instrument can be coupled to the prosthesis shaft 34, namely screwed thereto, in extension of the prosthesis shaft longitudinal axis 52.

As explained in detail above, such a handling instrument has the disadvantage that it is pressed to the side by surrounding tissue when being brought up to the internally threaded portion 52, such that a surgeon often has difficulties to screw the externally threaded portion of the handling instrument directly into the internally threaded portion 56 of the prosthesis shaft 34 without damaging the threaded portions.

To resolve this issue, adapter apparatus 32 is provided. It comprises a first adapter device 60 and a second adapter device 62.

The first adapter device 60 is configured to be releasably coupled to the shaft coupling element 54 in a force-locking and/or positive-locking manner in the coupling position depicted in FIG. 1. The second adapter device 62 is configured to releasably couple to the handling instrument 40. Furthermore, the second adapter device 62 is arranged or formed on the adapter apparatus 32 laterally offset relative to the first adapter device 60.

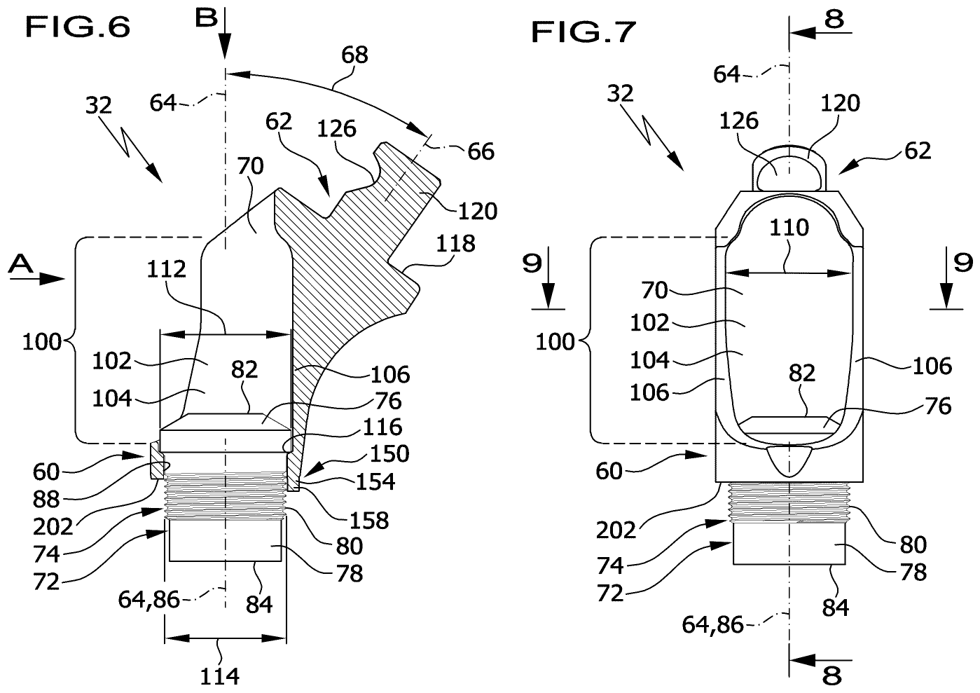

The first adapter device 60 defines a first adapter longitudinal axis 64. The second adapter device 64 defines a second adapter longitudinal axis 66. As schematically depicted in FIG. 6, the first adapter longitudinal axis 64 and the second adapter longitudinal axis 66 enclose an adapter angle 68. This configuration results in the laterally offset arrangement described above of the second adapter device 62 relative to the first adapter device 60, namely in particular relative to its first adapter longitudinal axis 64. In particular, the second adapter device 62 is not configured coaxially with the first adapter longitudinal axis 64, but instead is spaced laterally at a distance therefrom. This allows direct access to the first adapter device coming from the proximal direction.

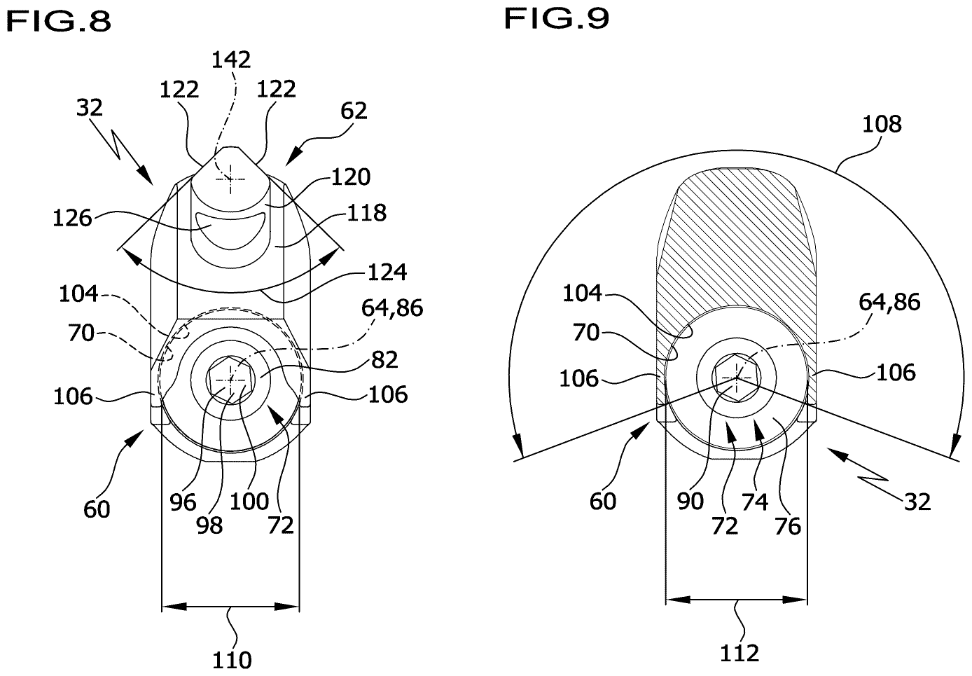

In the embodiment depicted in the Figures, the adapter angle 68 has a value in a range of about 15° to about 60°, namely in particular in a range of about 30° to about 45°. In the embodiment of the adapter apparatus 32 shown in FIGS. 1 to 9, the adapter angle 68 is about 35°.

The embodiment of the adapter apparatus 32 depicted in FIGS. 1 to 9 is configured in such a way that the first adapter device 60 comprises an adapter element receptacle 70 and an adapter element 72. The adapter element 72 is configured in the form of a screw element 74 with a head 76 and a screw shaft 78 extending in the distal direction therefrom. An externally threaded portion 80 is formed on the screw shaft 78. The adapter element has a proximal end 82 and a distal end 84 and defines an adapter element longitudinal axis 86. In the coupling position shown schematically in FIG. 1, the first adapter longitudinal axis 64 and the adapter element longitudinal axis 86 are aligned coaxially with one another, thus they coincide.

In the manner described, the first adapter device 60 comprises the externally threaded portion 80 corresponding to the internally threaded portion 56.

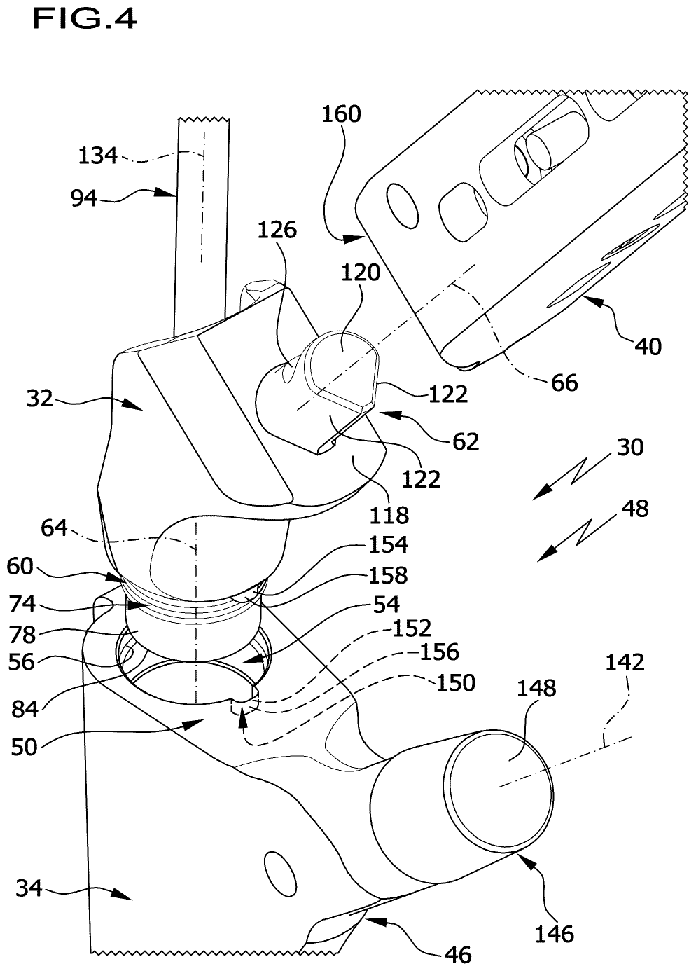

In a separating position, as shown schematically in FIG. 4, in which the adapter apparatus 32 and the prosthesis shaft 34 are out of engagement, the adapter element 72 is movable relative to the adapter element receptacle 70, namely displaceable in the direction of the adapter element longitudinal axis 86, and is also rotatable about said axis.

The adapter element receptacle 70 is formed on the adapter apparatus in the form of a perforation 88. The screw shaft 78 in the coupling position passes through the perforation 88, such that the distal end 84 of the adapter element 72 projects distally out of the adapter element receptacle 70 in the coupling position.

Arranged or formed on the head 76, thus at the proximal end 82 of the adapter element 72, is a tool element 90 pointing in the proximal direction for being brought into rotationally-fixed engagement with a distal end 92 of a screw-in tool 94. The tool element 90 is configured in the form of a tool element receptacle 96 open pointing in the proximal direction, and in the embodiment depicted in the Figures namely in the form of an internal polygon 98, specifically in the form of an internal hexagon. In alternative embodiments, the tool element receptacle 96 is configured in the form of an internal multilobular profile.

The adapter element 72 is held captive on the adapter apparatus 32 in the embodiment depicted in FIGS. 1 to 9. For this purpose, the adapter element receptacle 70 comprises a head receptacle portion 100, which proximally adjoins the perforation 88. The head receptacle portion 100 has an engagement opening 102, which is dimensioned such that the head 76 is movably held in the head receptacle portion 100.

The head receptacle portion 100 defines a receiving space 104 for the head 76. The engagement opening 102 is dimensioned such that the head 76 cannot pass through the engagement opening 102. This is achieved in that the head receptacle portion 100 comprises an outer delimiting wall 106, which extends over a circumferential angle of more than 180º relative to the first adapter longitudinal axis 64. The circumferential angle 108 shown as an example in FIG. 9 is about 225° and thus more than 200° in the case of the embodiment of FIG. 9.

To insert the adapter element 72 with the head 76 into the adapter element receptacle 76, the delimiting wall 106 must be widened, namely in such a way that an opening width 110, as it is schematically depicted in FIG. 7 and is smaller than an outer diameter 112 of the head 76, is dimensioned such that it corresponds to at least the outer diameter 112. After the insertion of the head 76 through the widened engagement opening 102, the delimiting wall 106 is then deformed back into the position shown in the Figures, such that the head 76 and thus the adapter element 72 is held captive in the adapter element receptacle 70 in a movable manner.

To prevent the head 76 from passing through the perforation 88, its outer diameter 112 is greater than an outer diameter 114 of the externally threaded portion 80. In order to delimit the movement of the adapter element 72 in the distal direction, an annular stop face 116 pointing in the proximal direction is formed on the head receptacle portion 100 in the transition region to the perforation 88.

The second adapter device 62 comprises an adapter end face 118 pointing away from the adapter apparatus 32 in the direction of the second adapter longitudinal axis 66 and comprises a coupling projection 120 projecting from the adapter end face 118. Said coupling projection has a cross sectional shape deviating from a circular shape in a section plane perpendicular to the second adapter longitudinal axis 66. The coupling projection 120 has a cylindrical basic shape, but is provided with two planar side faces 122, which enclose between them an angle 124 that is slightly smaller than 90°.

Furthermore, a coupling recess 126 that is open pointing away from the coupling projection 120 in the direction toward the first adapter longitudinal axis 64 is formed on the coupling projection 120 in a direction transverse relative to the second adapter longitudinal axis 66. The coupling recess 126 serves to accommodate a locking member 128 that is movably arranged on the handling instrument 40 in order to simply and quickly couple the handling instrument 40 to the second adapter device 62 in a force-locking and positive-locking manner. A basic structure and a corresponding coupling mechanism of the handling instrument 40 is described in detail, for example, in DE 10 2008 064 518 A1.

In the implantation system 30, a rasp adapter 130 is arranged or formed on the rasp shaft 42. The handling instrument 40 has a rasp adapter receptacle 132 that is configured corresponding to the rasp adapter 130. In order to for the adapter apparatus 32 or the rasp shaft 40 to be selectively coupleable to the handling instrument 40, the rasp adapter 130 is configured in such a way that it corresponds or substantially corresponds in its shape and size to the second adapter device 62 of the adapter apparatus 32. The coupling projection 120 and rasp adapter 130 are thus of identical configuration.

The screw-in instrument 94 is configured cooperating with the first adapter device 60 and defines a screw-in instrument longitudinal axis 134, also referred to as a screw-in tool longitudinal axis, namely with a screw-in instrument shaft 136.

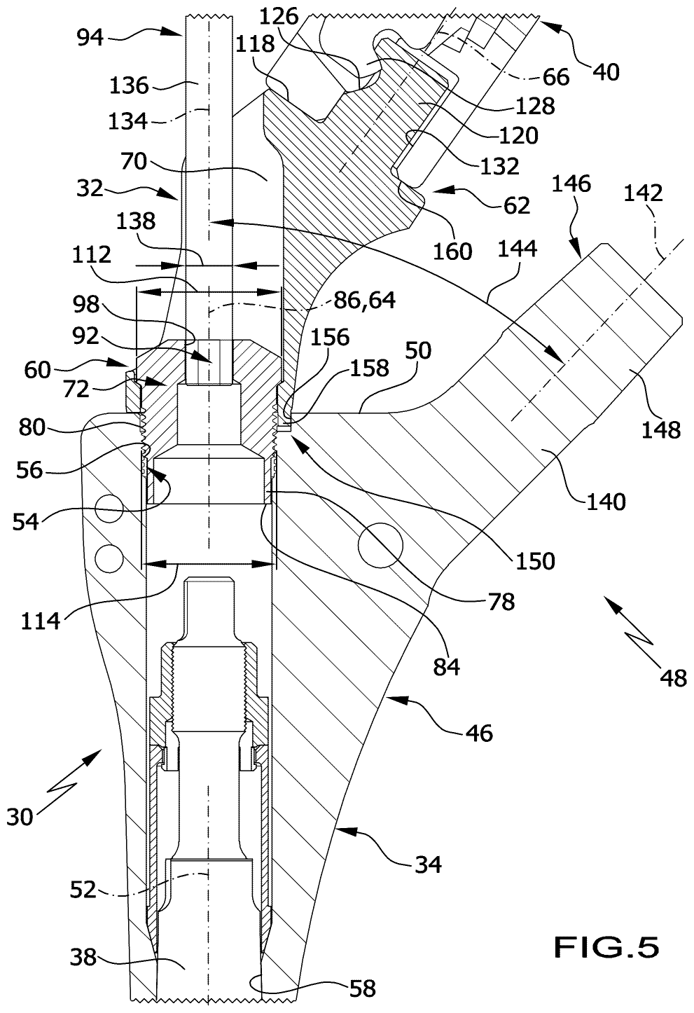

So that a surgeon can bring the screw-in instrument 94 with its distal end 92 into engagement with the tool element 90 and then in this way manipulate the adapter element 72, an outer diameter 138 of the screw-in instrument shaft 136 is significantly smaller than the outer diameter 114 of the externally threaded portion 80. As schematically illustrated in FIG. 5, the outer diameter 138 is at most half as large as the outer diameter 114. It is about one third of it. In the case of the insertion instrument not depicted in the Figures, the distal end thereof being provided with an externally threaded portion corresponding to the internally threaded portion 56, an outer diameter of the instrument shaft is approximately in the order of magnitude of the outer diameter 112 of the head 76. Such an instrument shaft is understandably more difficult to pass through body tissue than a slim, light screw-in instrument shaft 136 of the screw-in instrument 94.

Furthermore, formed at the proximal end 50 of the prosthesis shaft 34 is a laterally projecting prosthesis neck 140, which defines a prosthesis neck longitudinal axis 142. The prosthesis neck 140 is inclined relative to the prosthesis shaft longitudinal axis 52 by a neck angle 144. The neck angle has a value in a range of about 20° to about 60°.

A free end 146 of the prosthesis neck 140 is configured in the form of a coupling cone 148. It can be coupled to a joint head not depicted in the Figures or to a neck extension of the joint endoprosthesis.

The implantation system 30 further comprises an anti-rotation device 150 for holding the adapter apparatus 32 on the prosthesis shaft 34 in a rotationally-fixed manner in the coupling position. For this purpose, the anti-rotation device 150 comprises first and second anti-rotation elements 152 and 154.

The first anti-rotation element 152 is arranged or formed on the prosthesis shaft 34. The second anti-rotation element 154 is arranged or formed on the adapter apparatus 32. In the coupling position, the first and second anti-rotation elements 152, 154 are in engagement in order to prevent a rotation of the adapter apparatus 32 and the prosthesis shaft 34 relative to one another about the prosthesis shaft longitudinal axis 52.

The first anti-rotation element 152 is configured in the form of a depression 156, the second anti-rotation element 154 in the form of a projection that protrudes in the radial direction relative to the first adapter longitudinal axis 64.

The adapter apparatus 32 is of one-piece, namely monolithic configuration, except for the adapter element 72. It has an overall length which corresponds substantially to a length of the prosthesis neck 140, as can be easily seen in FIG. 5.

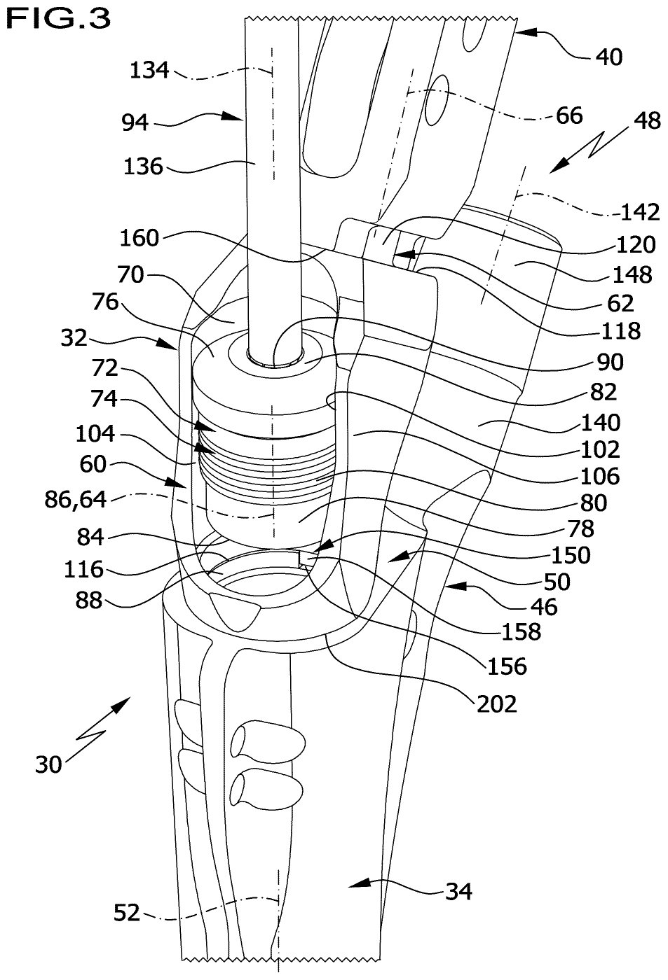

For extracting the prosthesis shaft 34 from the cavity of the bone in which the prosthesis shaft 34 is inserted, the adapter apparatus 32 is first connected to the shaft coupling element 54, namely in such a way that the adapter apparatus 32 is placed against the proximal end 50 of the prosthesis shaft 34, so that the first and second anti-rotation elements 152, 154 engage with one another. This is shown schematically in FIG. 3.

The distal end 92 of the screw-in instrument 94 can now be brought into engagement with the tool element 90, as is also schematically depicted in FIG. 3.

As is depicted in FIG. 5, the adapter element 72 is screwed so far into the prosthesis shaft 34 until the adapter apparatus 32 is held on the proximal end 50 of the prosthesis shaft 34 in a clamping manner. Now, as is schematically indicated in FIG. 4, a distal end 160 of the handling instrument 40 can be brought up to the coupling projection 120 and the latter can be accommodated in the rasp adapter receptacle 132. The locking member 128 on the handling instrument 40 is pivoted into the coupling recess 126. The distal end 160 is hereby supported on the adapter end face 118. The handling instrument 40 is now, as described and schematically illustrated in FIGS. 1 to 3 and 5, coupled to the adapter apparatus 32 in a force-locking and positive-locking manner.

A strike plate 162 is formed at the proximal end of the handling instrument 40. By acting upon the strike plate 162 with strikes directed in the proximal direction, for example with a hammer or the like, the prosthesis shaft 34 can now be extracted from the cavity of the bone of the patient.

To insert the prosthesis shaft into the cavity of the bone, the procedure can be reversed. To this end, the prosthesis shaft 34 can be coupled to the adapter apparatus 32 and the handling instrument 40 outside of the body of the patient.

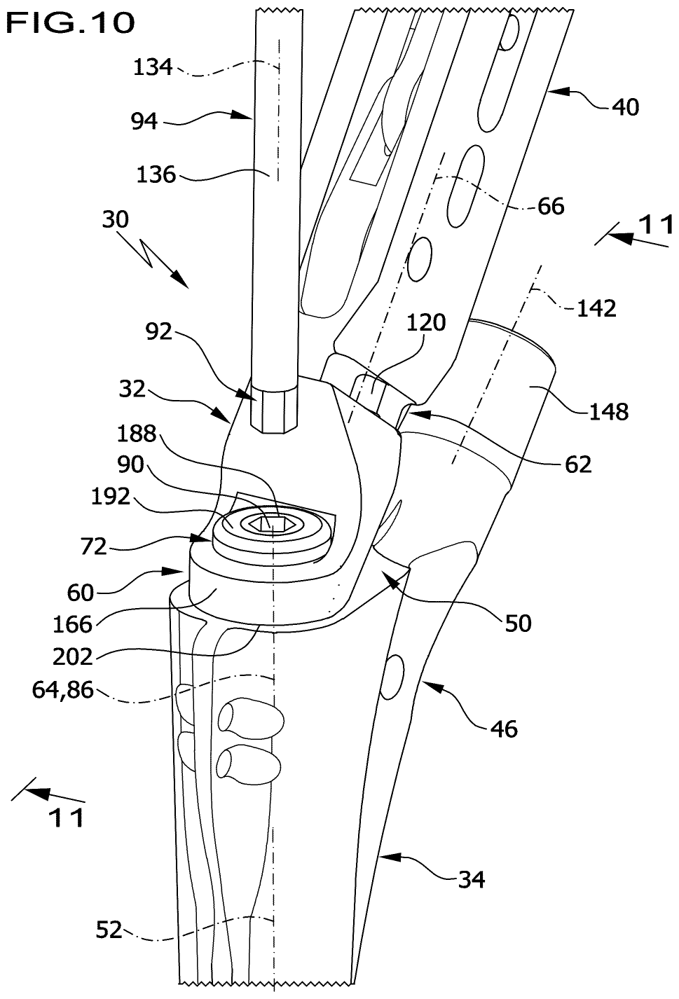

Schematically depicted as an example in FIGS. 10 to 14 is a further embodiment of an implantation system 30. It differs from the embodiment of the implantation system 30 according to FIGS. 1 to 9 in the configuration of the adapter apparatus 32. Therefore, for reasons of clarity, the same reference numerals are used in FIGS. 10 to 14 for identical components or components comparable in their function as in the embodiment of FIGS. 1 to 9.

In the case of the adapter apparatus 32 according to FIGS. 10 to 14, no anti-rotation device 150 is provided.

In this embodiment, the second adapter device 62 is configured identically to the second adapter device 62 in the embodiment of FIGS. 1 to 9, such that reference can be made to the above description in this regard, in particular in regard to the coupling with the handling instrument 40.

In this embodiment, too, the adapter element 72 is held captive on the adapter apparatus 32. This is achieved by an undercut 164 on the adapter element 72. A retaining projection 166 of the adapter apparatus 32 cooperates with this undercut 164, said retaining projection engaging into the undercut 164.

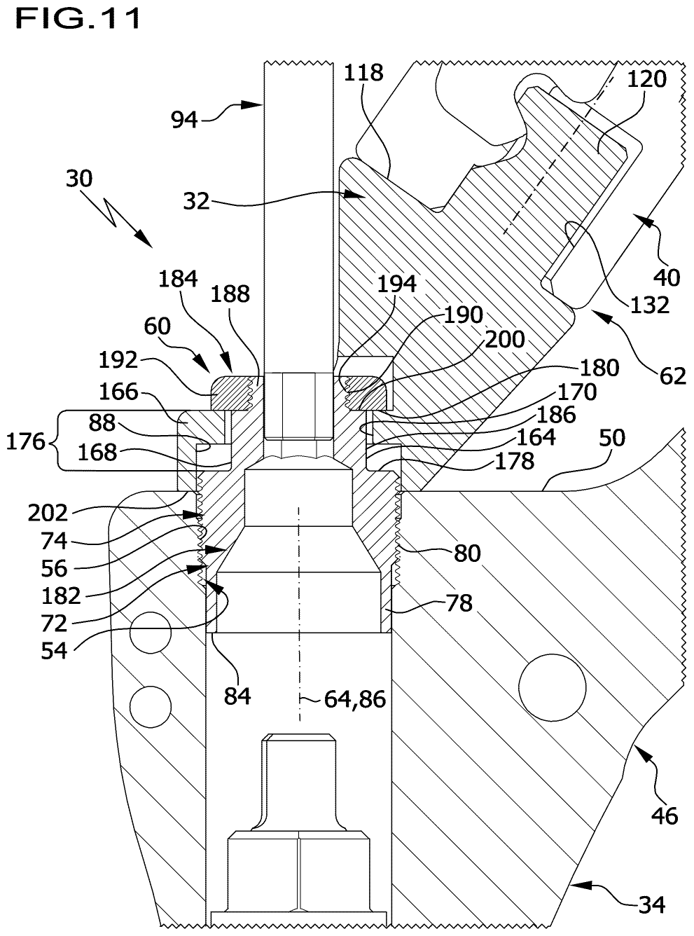

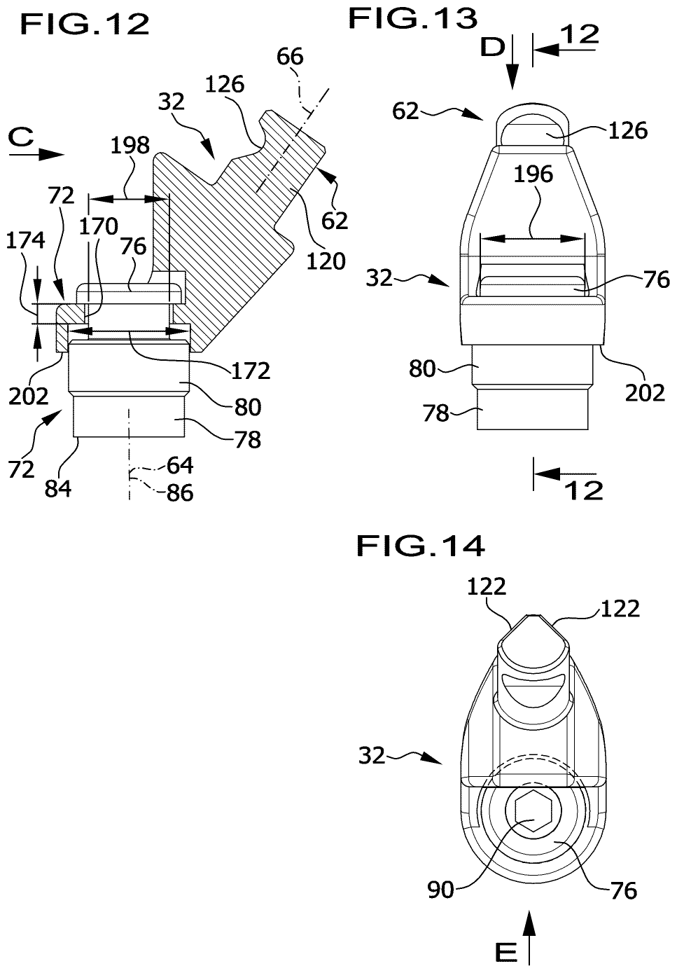

As schematically illustrated in FIGS. 11 and 12, the undercut 164 is configured in the form of an annular groove 168 that is open in the radial direction relative to the adapter element longitudinal axis 86. The retaining projection 166 is configured in the form of an annular flange 170 pointing in the radial direction toward the adapter element longitudinal axis 86. The annular flange 170 is created on the adapter apparatus 32 by a one-step reduction of an inner diameter 172 of the perforation 88, which extends in the proximal direction commencing from the distal end 84 of the adapter apparatus 32.

The annular flange 170 has a width 174 in parallel to the adapter element longitudinal axis 86 that is smaller than a width 176 of the annular groove 168. This makes it possible to displace the adapter element 72 relative to the adapter apparatus 32 in parallel to the adapter element longitudinal axis 86, namely either until an annular face 178, which points in the proximal direction and delimits the annular groove 168, strikes against the annular flange 170, or an annular face 180 pointing in the distal direction, which proximally delimits the annular groove 168. FIGS. 11 and 12 show the annular face 180 abutting against the annular flange 170, i.e., the most distal position of the adapter element 72 relative to the adapter apparatus 32.

In order to mount the adapter element 72 on the adapter apparatus 32 in a simple manner, the adapter element 72 is of multi-part, namely two-part configuration, and comprises a first adapter element part 182 and a second adapter element part 184. These are configured cooperating in such a way that the undercut 164, thus the annular groove 168, is delimited both by the first adapter element 182 and by the second adapter element 184. In addition, the adapter element parts 182 and 184 are connected to one another in a non-releasable manner. For example, they may be adhesively bonded or welded to one another.

The first adapter element part 182 forms substantially the entire adapter element 72. It comprises the annular face 178 and a cylindrical delimiting face 186 pointing away from the adapter element longitudinal axis 86, said delimiting face extending between the two annular faces 178 and 180. On the proximal side of the delimiting face 186, an outer diameter 198 of the first adapter element part 182 decreases in one step, such that a connecting portion 188 forms a proximal end of the first adapter element part 182. Said connecting portion 48 is provided with an external thread 190.

The second adapter element part 184 is configured substantially in the form of a nut 192, which comprises an internal thread 194 corresponding to the external thread 190. An outer diameter 196 of the nut 192 is greater than the outer diameter 198 of the delimiting face 186, such that the second adapter element part 184 forms the annular face 180.

For mounting the adapter apparatus 32, the first adapter element part 182 is guided with the connecting portion 188 in the proximal direction through the perforation 88 until the connecting portion 188 projects proximally beyond the annular flange 170. In this position, the nut 192 can then be screwed to the connecting portion 188, namely so far until it abuts against an annular face 200 that connects the connecting portion 180 and the delimiting face 166 and points in the proximal direction. In this position, as shown schematically in FIG. 11, the connection between the two adapter element parts 182 and 184 can then be secured by adhesive bonding or welding.

The handling of the implantation system 30 with the embodiment of the adapter apparatus 32 according to FIGS. 10 to 14 corresponds to the handling of the implantation system 30 according to FIGS. 1 to 9, such that reference can be made to the above description in its entirety in this regard.

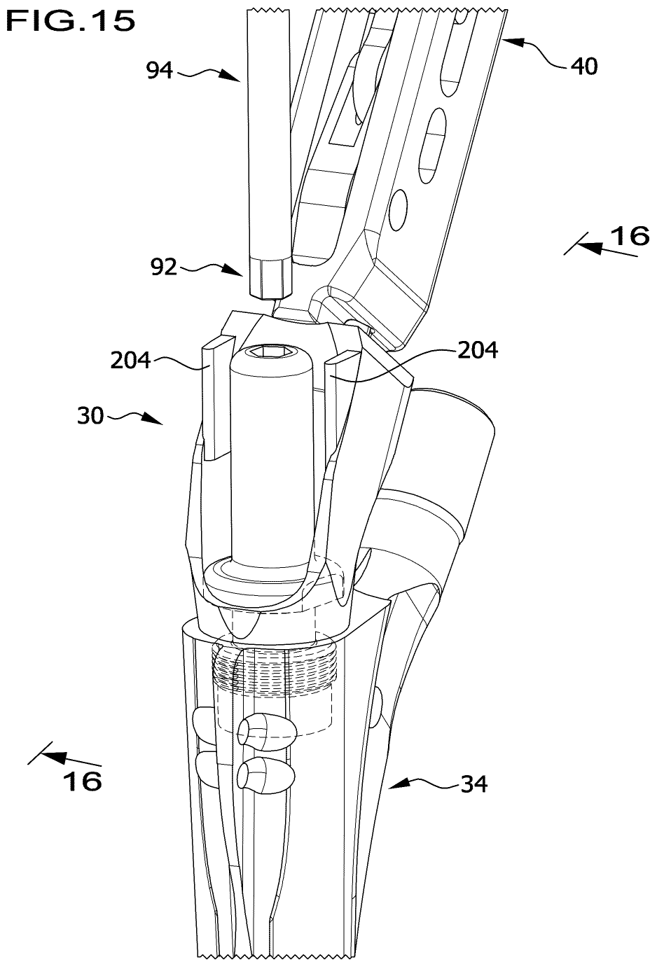

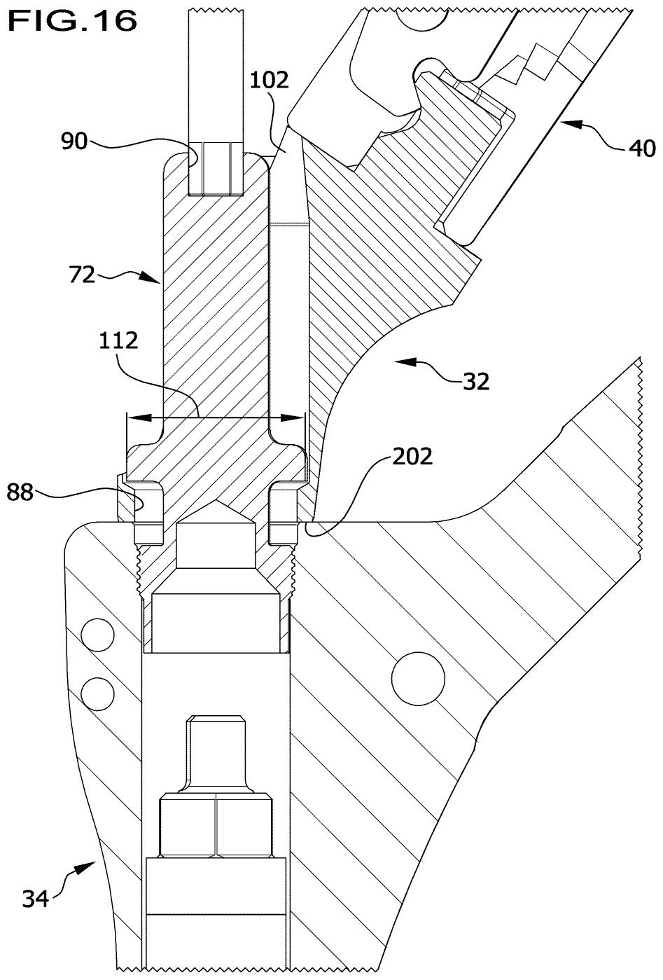

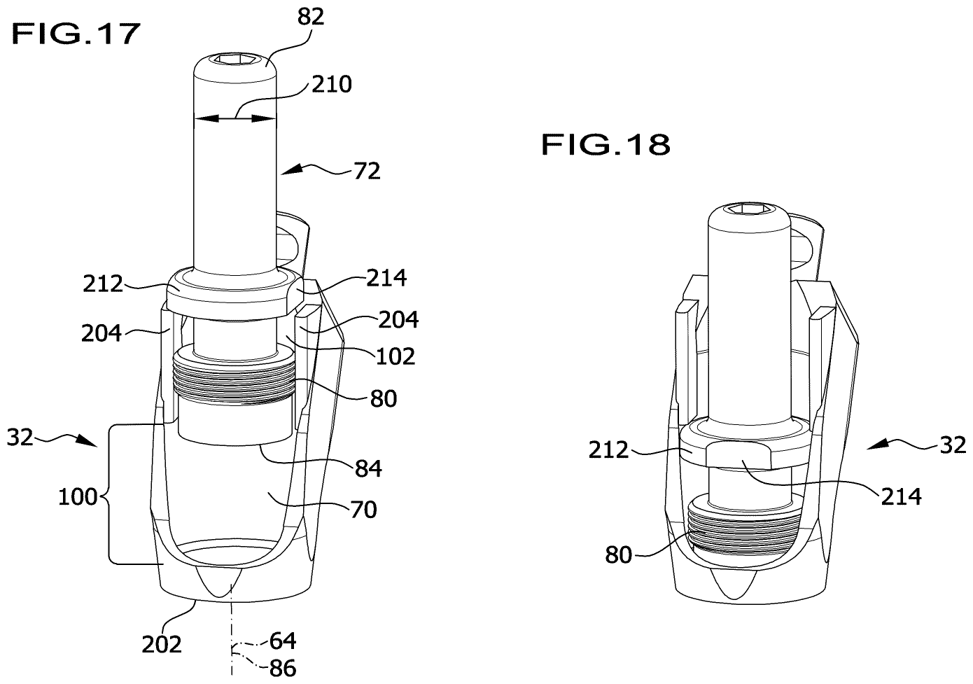

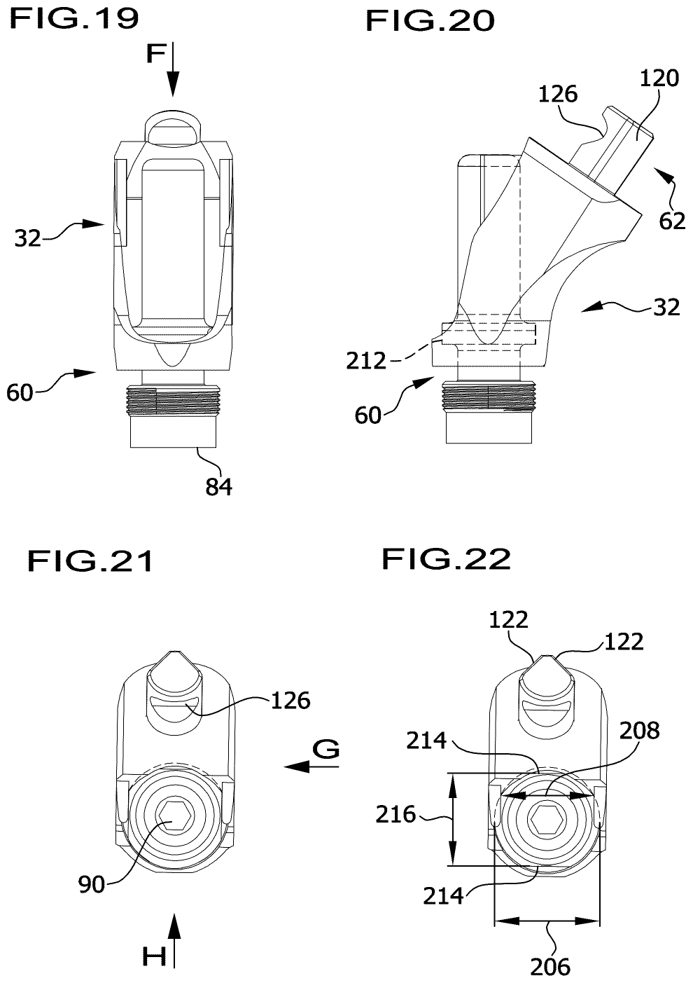

A further embodiment of an implantation system 30 is schematically depicted as an example in FIGS. 15 to 22. It differs only in the design of the adapter device 32 from the embodiments of FIGS. 1 to 9 and 10 to 14, so that only the differences of the adapter apparatus 32 in comparison to the adapter apparatus 32 of the aforementioned embodiments are explained below. Here, too, the same reference numerals are used to identify identical or functionally similar components.

The adapter apparatus 32 is equipped with a second adapter device 62, which is identical to the second adapter devices 62 of the previously described embodiments, such that reference can be made thereto in full in this regard.

There are differences in the design of the first adapter device 60. It comprises an adapter element 72 and an adapter element receptacle 70.

The adapter element receptacle 70 comprises a perforation 88 and a head receptacle portion 100 adjoining said perforation on the proximal side. The perforation 88 extends in the proximal direction commencing from a distal end 202 of the adapter apparatus 32.

In the region of a proximal end of the head receptacle portion 100, two cuboidal constriction bodies 204 are arranged diametrically opposed to one another relative to the first adapter longitudinal axis 64. On the distal side of the constriction bodies 204, the head receptacle portion 100 defines a free inner diameter 206. A distance 208 between the two constriction bodies 204 is smaller than the inner diameter 206.

The adapter element 72 is configured substantially in the form of an elongated cylinder, which defines an outer diameter 210 commencing from the proximal end 82. The shaft of the adapter element 72 expands in the distal direction in one step on about a final fourth of its total length and there is provided with the externally threaded portion 80, which corresponds to the internally threaded portion 56 of the prosthesis shaft 34.

On the shaft portion of the adapter element 72 with the outer diameter 210, a head-like annular flange 212 is formed somewhat at a distance from the externally threaded portion 80, said annular flange having an outer diameter that is slightly smaller than the inner diameter 206. Furthermore, two flattened portions 214 pointing diametrically away from one another relative to the first adapter longitudinal axis 64 are formed on the annular flange 212, said flattened portions having a distance 216 from one another that is slightly smaller than the distance 208.

As a result of the described configuration of the adapter apparatus 32 and the adapter element 72, it is possible to completely separate them from one another. However, the adapter element 72 can be coupled to the adapter apparatus 32 by aligning the flattened portions 214 in the direction pointing toward the constriction bodies 204 and then inserting the adapter element 72 with its distal end 84 in front coming from the proximal side in the distal direction into the adapter element receptacle 70. In the described orientation, the annular flange 212 can be pushed past the constriction bodies 204.

As soon as the annular flange 212 is positioned on the distal side of the two constriction bodies 204, the adapter element 72 can be rotated about the first adapter longitudinal axis 64. In the event that the flattened portions 214 are not oriented in parallel to mutually facing side faces of the constriction bodies 204, the adapter element 72 cannot be pulled out of the adapter element receptacle 70 in the proximal direction. In the manner described, therefore, due the special design of the adapter element 72 and the adapter apparatus 32, a coupling according to the key-keyhole principle is thus achieved by engaging the head-like or head-forming annular flange 212. The adapter element 72 and the adapter apparatus 32 are thus configured in such a way that the annular flange 212 is insertable in a direction parallel to the first adapter longitudinal axis 64 into the adapter element receptacle 70, i.e. through engagement opening 102, into the head receptacle portion 100 only in a defined rotational position relative to the first adapter longitudinal axis 64. The head receptacle portion 100 defines between the two constriction bodies 204 the engagement opening 102, which thus defines a non-round inner cross section. An outer contour of the annular flange 212 is configured corresponding to the non-round inner cross section and thus is also non-round. Here, non-round means deviating from a circular shape.

For coupling to a distal end 92 of the screw-in instrument 94, in an analogous manner as described above, a tool element 90 pointing in the proximal direction is formed on the adapter element 72.

Regarding the functioning of the implantation system 30, as in the other embodiments, reference can be made to the above detailed description of the embodiment of FIGS. 1 to 9 in its entirety.

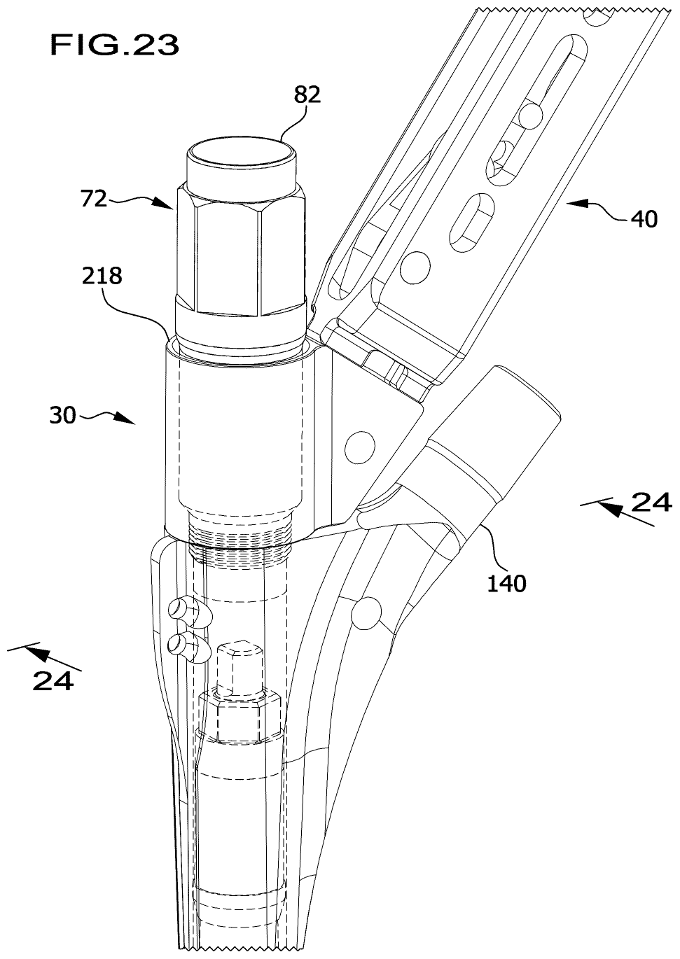

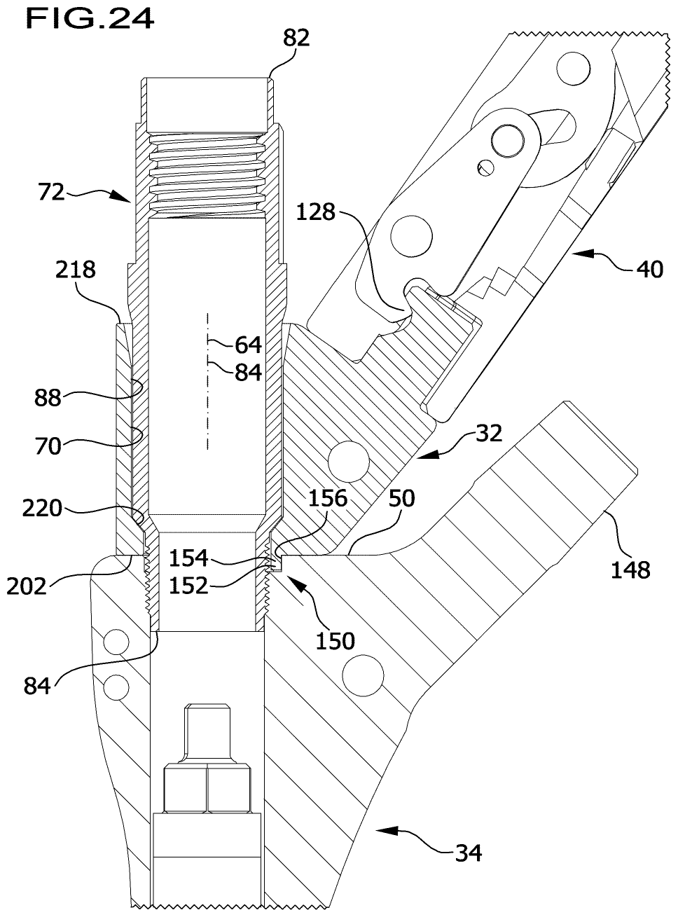

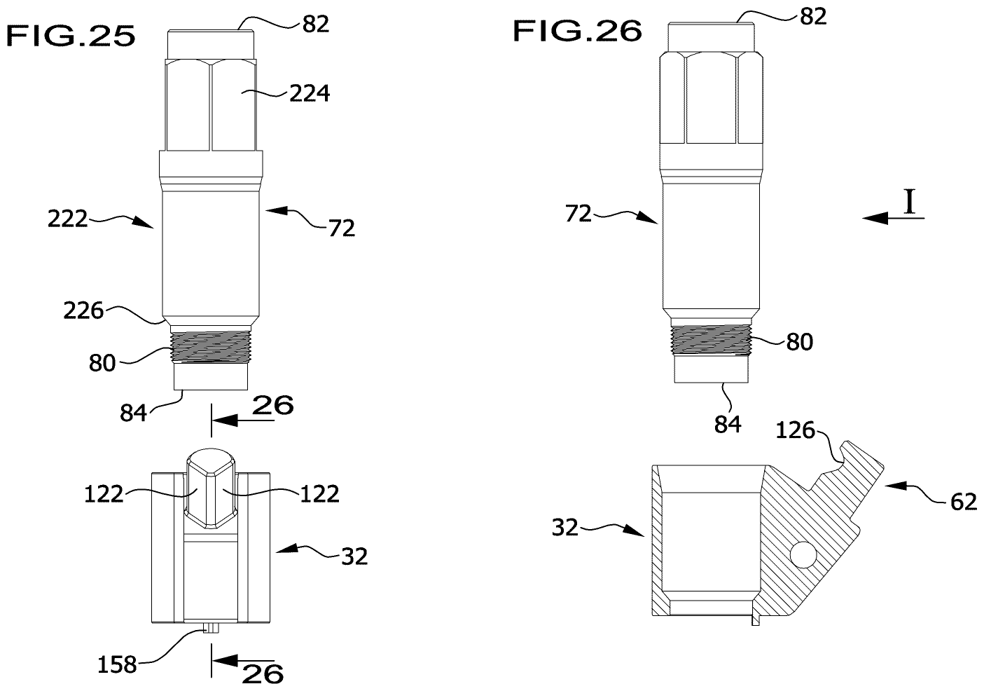

A further embodiment of an implantation system is depicted as an example in FIGS. 23 to 26. It differs from the embodiments of implantation systems 30 described above only in the design of the adapter apparatus 32.

In the case of this adapter apparatus 32, too, the second adapter device 62 is of identical configuration as in the embodiments described above. Here, too, identical or functionally similar components are provided with the same reference numerals to increase clarity.

The adapter element receptacle 70 in this embodiment is configured as part of the first adapter device 60 in the form of an elongated hollow-cylindrical perforation 88, which tapers in inner diameter at its distal end toward a distal end 202 of the adapter apparatus 32, such that an inclined annular stop face 270 pointing in the direction toward the first adapter longitudinal axis 64 is formed.

From the distal end 202, a projection 158 protrudes in the distal direction, which forms a second anti-rotation element 154 of an anti-rotation device 150. A first anti-rotation element 152 is configured in the form of a depression 156 on the prosthesis shaft 34. The adapter apparatus 32, as explained above in conjunction with the embodiment of FIGS. 1 to 9, is coupled to the prosthesis shaft 34 in a rotationally-secured manner.

The adapter element 72 has a length, namely such that the proximal end 82 of the adapter element 72 projects proximally out of the adapter element receptacle 70, in particular beyond the proximal end 218 of the adapter apparatus 32, when the adapter apparatus 32 is coupled to the prosthesis shaft 34.

In this embodiment, the adapter element 72 is formed by part of a separating instrument 222 comprised by the implantation system 30, namely an inner screw of said separating instrument. Thus, instead of a special adapter element 72, as is provided in the embodiments of the implantation systems 30 described above, a component can be used that is required anyway for extracting a prosthesis shaft 34.

In this embodiment, the adapter element 72 and the adapter apparatus 32 can be completely separated from one another. They are configured without a securing device; thus, they cannot be secured to one another in a captive manner.

Commencing from its proximal end 82, the adapter element 72 has an external polygon 224, which in the coupling position projects proximally beyond the proximal end 218 of the adapter apparatus 32. This allows a surgeon to grasp the adapter element 72 in the region of the external polygon 224 without the aid of a screw-in instrument 94 and to screw the externally threaded portion 80 formed in the region of a distal end 84 of the adapter element 72 to the internally threaded portion 56 of the prosthesis shaft 34.

In order to prevent the adapter element 72 from passing through the perforation 88, an outer diameter of the adapter element 72 increases somewhat on the proximal side of the externally threaded portion 80, such that an inclined annular face 226 corresponding to the stop face 220 is formed, which in the coupling position abuts against the stop face 222 in order to thus push the adapter apparatus 32 against the prosthesis shaft 34 in a clamping manner.

Regarding the handling of the implantation system 30, reference can be made to the above description in conjunction with FIGS. 1 to 9 in its entirety.

All embodiments of implantation systems 30 described above make it possible, in particular, to use the handling instrument 40 both as a handle for the rasp shaft 42 to form a rasp 44 and for coupling to the adapter apparatus 32, in particular to the second adapter device 62. Since during a surgical procedure for inserting a prosthesis shaft 34 into the cavity of a bone, the cavity cannot simultaneously be prepared and the prosthesis shaft 34 inserted, one single handling instrument 40 is sufficient to handle the rasp shaft 42 on the one hand and the prosthesis shaft 34 on the other hand.

The coupling to the prosthesis shaft 34 is made possible by the adapter apparatus 32, which in the embodiments described above has a second adapter device 62 that is configured identically to the rasp adapter 130 on the rasp shaft 42 for coupling to the handling instrument 40. Thus, the instruments required for a surgical procedure for inserting a prosthesis shaft 34 can be reduced to a minimum. This significantly increases clarity during surgery.

### REFERENCE NUMERAL LIST

30 implantation system
32 adapter apparatus
34 prosthesis shaft
36 distal end
38 shaft extension
40 handling instrument
42 rasp shaft
44 rasp
46 femoral shaft
48 hip joint endoprosthesis
50 proximal end
52 prosthesis shaft longitudinal axis
54 shaft coupling element
56 internally threaded portion
58 longitudinal perforation
60 first adapter device
62 second adapter device
64 first adapter longitudinal axis
66 second adapter longitudinal axis
68 adapter angle
70 adapter element receptacle
72 adapter element
74 screw element
76 head
78 screw shaft
80 externally threaded portion
82 proximal end
84 distal end
86 adapter element longitudinal axis
88 perforation
90 tool element
92 distal end
94 screw-in instrument
96 tool element receptacle
98 internal polygon
100 head receptacle portion
102 engagement opening
104 receiving space
106 delimiting wall
108 circumferential angle
110 opening width
112 outer diameter
114 outer diameter
116 stop face
118 adapter end face
120 coupling projection
122 side face
124 angle
126 coupling recess
128 locking member
130 rasp adapter
132 rasp adapter receptacle
134 screw-in instrument longitudinal axis
136 screw-in instrument shaft
138 outer diameter
140 prosthesis neck
142 prosthesis neck longitudinal axis
144 neck angle
146 free end
148 coupling cone
150 anti-rotation device
152 first anti-rotation element
154 second anti-rotation element
156 depression
158 projection
160 distal end
162 strike plate
164 undercut
166 retaining projection
168 annular groove
170 annular flange
172 inner diameter
174 width
176 width
178 annular face
180 annular face
182 first adapter element part
184 second adapter element part
186 delimiting face
188 connecting portion
190 external thread
192 nut
194 internal thread
196 outer diameter
198 outer diameter
200 annular face
202 distal end
204 constriction body
206 inner diameter
208 distance
210 outer diameter
212 annular flange
214 flattened portion
216 distance
218 proximal end
220 stop face
222 separating instrument
224 external polygon
226 annular face

1. An implantation system comprising a prosthesis shaft for insertion into a cavity of a bone, wherein the prosthesis shaft has a proximal end and a distal end and defines a prosthesis shaft longitudinal axis, wherein a shaft coupling element is formed at the proximal end for coupling to a first handling instrument for inserting the prosthesis shaft into the cavity and/or for extracting the prosthesis shaft from cavity, wherein the implantation system comprises an adapter apparatus with a first adapter device and a second adapter device, wherein the first adapter device is configured to releasably couple to the shaft coupling element in force-locking and/or positive-locking manner in a coupling position, wherein the second adapter device is configured to releasably couple to a second handling instrument, and wherein the second adapter device is arranged or formed on the adapter apparatus laterally offset relative to the first adapter device.

2. The implantation system according to claim 1, wherein the first adapter device defines a first adapter longitudinal axis, wherein the second adapter device defines a second adapter longitudinal axis, and wherein the first adapter longitudinal axis and the second adapter longitudinal axis enclose an adapter angle.

3. The implantation system according to claim 2, wherein the shaft coupling element comprises an internally threaded portion, and wherein the first adapter device comprises an externally threaded portion corresponding to the internally threaded portion.

4. The implantation system according to claim 3, wherein the first adapter device comprises an adapter element receptacle and an adapter element, and wherein in a separating position, in which the adapter apparatus and the prosthesis shaft are out of engagement, the adapter element is movable relative to the adapter element receptacle.

5. The implantation system according to claim 4, wherein the adapter element has a distal end and a proximal end and defines an adapter element longitudinal axis, and wherein in the coupling position the first adapter longitudinal axis and the adapter element longitudinal axis are oriented coaxially with one another.

6. The implantation system according to claim 4, wherein the adapter element comprises a screw element, and wherein the externally threaded portion corresponding to the internally threaded portion is formed on the adapter element commencing from its distal end.

7. The implantation system according to claim 4, wherein the adapter element is held captive on the adapter apparatus.

8. The implantation system according to claim 4, wherein the adapter element has an undercut and wherein a retaining projection of the adapter apparatus engages into the undercut.

9. The implantation system according to claim 4, wherein the adapter element has a head that forms the proximal end of the adapter element.

10. The implantation system according to claim 9, wherein an outer diameter of the head is larger than an outer diameter of the externally threaded portion.

11. The implantation system according to claim 10, wherein the adapter element receptacle has a head receptacle portion with an engagement opening and the engagement opening is dimensioned such that the head is movably held in the head receptacle portion.

12. The implantation system according to claim 11, wherein the head and the head receptacle portion are configured in such a way that the head is insertable in a direction parallel to the first adapter longitudinal axis through the engagement opening into the head receptacle portion only in a defined rotational position relative to the first adapter longitudinal axis.

13. The implantation system according to claim 1, wherein the implantation system comprises an anti-rotation device for holding the adapter apparatus on the prosthesis shaft in a rotationally fixed manner in the coupling position.

14. The implantation system according to claim 1, wherein the second adapter device comprises an adapter end face pointing away from the adapter apparatus in a direction of the second adapter longitudinal axis and a coupling projection projecting from the adapter end face.

15. The implantation system according to claim 1, wherein the first adapter longitudinal axis extends in parallel to the prosthesis shaft longitudinal axis or is defined by the prosthesis shaft longitudinal axis.

16. The implantation system according to claim 1, wherein the implantation system comprises a first handling instrument in the form of a driving-in and driving-out instrument with an instrument shaft.

17. The implantation system according to claim 1, wherein the implantation system comprises a rasp for preparing the cavity.

18. The implantation system according to claim 17, wherein the rasp is configured in such a way that it corresponds or substantially corresponds in its shape and size to the second adapter device.

19. The implantation system according to claim 1, wherein the implantation system comprises a screw-in instrument that cooperates with the first adapter device, said screw-in instrument having a screw-in instrument shaft defining a screw-in tool longitudinal axis.

20. The implantation system according to claim 1, wherein the prosthesis shaft is a femoral shaft of a hip joint endoprosthesis.
